(19) United States
(12) Patent Application Publication
(10) Pub. No.: US 2002/0164635 A1
(43) Pub. Date: Nov. 7, 2002

(54) Novel methods of directed evolution

(75) Inventor: John C. Salerno, Troy, NY (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Rensselaer Polytechnic Institute

(21) Appl. No.: 10/138,183

(22) Filed: May 2, 2002

Related U.S. Application Data
(60) Provisional application No. 60/288,527, filed on May 3, 2001.

(51) Int. Cl. C12Q 1/68; C07H 21/04; C12P 19/34

(52) U.S. Cl. 435/6; 435/91.2; 536/23.2

(57) ABSTRACT

Methods for generating chimeric polynucleotides by directed evolution are described. In the methods, splice points of interest are identified within the polynucleotides of a basis set of polynucleotides, preferably through the use of an algorithm that defines the number of splice points and selects the splice points, either by random selection or using information regarding alignment of the polynucleotides. The algorithms can include additional factors, including a definition of a desired distance between splice points, and/or weighing factors to bias selection of splice points. Chimeric polynucleotides are generated using primers (e.g., double primers or non-overlapping primers) and polymerase chain reaction or combinatorial strategies.

### RELATED APPLICATIONS

This application claims the benefit of U.S. Provisional Application No. 60/288,527, filed May 3, 2001. The entire teachings of the above application are incorporated herein by reference.

### BACKGROUND OF THE INVENTION

Production of proteins with novel properties has been a goal of the biotechnology industry and the basic life science research community for several decades. Proteins to be engineered include enzymes (engineered for novel chemistries, substrate specificities, altered solubility or altered stability); receptors; antibodies (engineered for altered ligand recognition); DNA binding proteins (engineered to recognize new sites or to provide signals of events inside the cell); and other proteins. Two major paths to the desired end are rational design and directed evolution.

One type of rational design includes de novo approaches in which a sequence not directly related to existing protein is specified and synthesized to produce a folded entity. The knowledge of protein folding, however, is insufficient for the practical production of novel proteins. Another approach for rational design uses existing proteins and incorporating specific alterations (e.g., modifications of amino acid residues to alter substrate or cofactor specificity). For example, a successful though limited approach is the production of fusion proteins in which two or more genes are combined in frame to produce a protein in which the regions coded for by the parent genes independently fold but are joined by a linking region.

The introduction of directed evolution methods to the problems of protein and pathway design has attracted considerable attention and excitement in the last decade. While rational protein design has made progress, the idea of using a method based on natural selection to develop new enzymes and structures has great appeal. Initial methods of directed evolution were based on cycles of mutagenesis and selection (see, e.g., Shao, Z. and Arnold, F. H., Curr. Opin. Struct. Biol. 6(4):513-8 (1996)). Although successes were recorded using this strategy, many attempts to evolve enzymes with desired characteristics were failures for reasons which were not always well understood. Furthermore, in directed evolution, as in natural selection, a pathway from the starting material to the desired resultant material must exist in which all the intermediates are reasonably successful. A weakness in this procedure is the need to proceed in very small jumps, restricting the volume of evolutionary space that is accessible.

More recently, methods loosely termed “gene shuffling” have been attempted (see, e.g., Crameri, A., et al., Nature 391(6664):288-291 (1998)). Initially, a basis set of homologous genes was restricted and the fragments randomly ligated. Most of products in such a protocol were nonsense DNA, but in a small minority of the cases, homologous fragments of related genes were ligated in the correct order. By applying selection criteria to a host transformed with the mixed DNA, a relatively small number of chimeras with desirable new features could be identified. A chimeric gene (or gene product) contains regions derived from two or more parent genes; to have a reasonable chance of stable folding, chimeric proteins were derived from genes composed of fragments from a basis set of related genes combined in frame and in order. This method allowed production of stably folded chimera which differ from the basis genes by more than a few point mutations, and provided additional evolutionary pathways that were not generally accessible by natural evolution. However, only a very small percentage of fragments were produced which had the potential to fold stably and have the desired activity. Furthermore, the number of potential chimeras which make up a region of evolutionary space spanned by a basis set are enormous.

Introduction of the polymerase chain reaction (PCR) into methods of directed evolution (see, e.g, Crameri, A., et al., Nature 391(6664):288-291 (1998); Newton, C. R. and Graham, A., PCR (BIOSis Scientific Publishers, Oxford, U.K., 1994); and Pelletier, J. N., Nat. Biotechnol. 19(4):314-5 (2001)) allowed ordered connection of related DNA fragments at natural splice sites. However, because fragments must prime each other with reasonable melting and annealing temperatures, splices between two genes occur only in regions of high similarity, as they require sufficient relatedness to allow mutual priming. Furthermore, methods for producing and screening all possible chimera are not yet known. A need remains for a method to sample evolutionary space in a productive way.

### SUMMARY OF THE INVENTION

The present invention is drawn to methods of generating chimeric polynucleotides, for purposes including directed evolution. The methods comprise generation of a prespecified set of chimeric polynucleotides, which can be facilitated by prior in silico gene shuffling. In the methods, a basis set of polynucleotides comprising three or more different polynucleotides is used. In one embodiment, at least two of the polynucleotides of the basis set have sufficient homology to one another to anneal for priming. One or more of the polynucleotides of the basis set can comprise whole genes; alternatively, none of the polynucleotides of the basis set can comprise whole genes. If desired, one or more of the polynucleotides of the basis set can include synthetic nucleic acids, and/or can incorporate one or more non-native splice points.

Splice points of interest are identified within the polynucleotides of the basis set, wherein each polynucleotide in the basis set has the same number of splice points. The splice points can be identified by use of an algorithm that defines the position of naturally occurring splice points (defined by regions of homology sufficient to allow fragments to prime each other). For synthesis methods which do not depend on natural homology, splice points can be identified by random selection; alternatively, they can be identified using information regarding alignment of the polynucleotides. Algorithms can include additional factors, including a definition of a desired distance between splice points, and/or weighing factors to bias selection of splice points, such as weighing factors that bias selection of splice points in regions of interest in the polynucleotides of the basis set; that bias selection of splice points in regions having a preselected percentage of homology among the polynucleotides of the basis set; and/or bias selection of splice points in structurally identifiable regions of the polypeptides encoded by the polynucleotides of the basis set.

In one embodiment, double primers are used to generate the chimeric polynucleotides. Oligonucleotide double primer sets are created for each splice point, in which each double primer in a set comprises a “pre” region joined to and followed immediately by a “post” region. The “pre” region comprises an oligonucleotide primer for a splice point in one polynucleotide in the basis set, and the “post” region comprises an oligonucleotide primer for the complement of the corresponding splice point in another polynucleotide in the basis set. The set of double primers includes double primers comprising all possible combinations of pre and post regions for each splice point. The double primer sets are used in the polymerase chain reaction to amplify combinations of fragments, thus generating a multitude of chimeric polynucleotides, in which each chimeric polynucleotide comprises a fragment from at least two of the polynucleotides in the basis set.

In another embodiment, when the splice points of interest within the polynucleotides of the basis set are identified, the splice points divide each polynucleotide into M consecutive fragments in a correct order. Non-overlapping oligonucleotides are generated for each fragment of the M fragments for each polynucleotide in the basis set; these oligonucleotides are not primers, since they have no overlap and do not anneal, but instead are combinatorially combined (e.g., by ordered ligase reactions). Oligonucleotides corresponding to consecutive fragments are ligated in the correct order to generate a multitude of correctly ordered chimeric polynucleotides, in which each chimeric polynucleotide comprises a fragment from some, or all, of the polynucleotides in the basis set. In one embodiment, pairs of oligonucleotides corresponding to two consecutive fragments are ligated to generate dimers, and the dimers are subsequently ligated consecutively, to generate correctly ordered chimeric polynucleotides.

In either method, the resultant polypeptides comprise fragments from at least two of the polynucleotides in the basis set; in one embodiment, the chimeric polynucleotides comprise polynucleotides comprising a fragment from each polynucleotide in the basis set. If desired, a solid phase can be used during generation of the polynucleotides, so that the chimeric polynucleotides are attached to a solid phase. Additional steps can be included to limit production of certain chimeric polynucleotides in favor of other chimeric polynucleotides: for example, one or more “polishing” steps can be included during polymerase chain reaction, in which loose single stranded ends of products are briefly digested with an exonuclease. In another example, one or more “poisoned primers” can be used, where the poisoned primers hybridize with high stringency to an product which is incapable of supporting polymerase chain reaction, thereby interrupting extension during polymerase chain reaction.

The methods describe herein allow flexible generation of novel chimeric polynucleotides, from which polypeptides can be prepared. The methods provide a productive sample of evolutionary space for the polynucleotides in the basis set, and allow use of polynucleotides in the basis set that are not closely homologous, thereby producing chimeric polynucleotides previously unavailable by traditional modes of directed evolution.

### BRIEF DESCRIPTION OF THE DRAWINGS

FIG. 1 is a representation of a table demonstrating pairwise values of melting temperature T(n, m,k,l) between polynucleotides n and m of a basis set, for nucleotide fragments beginning at position k and extending l bases. Each pair is represented as an element (e.g., A1); hybridization of each element (e.g., A1) with the desired melting temperature to other elements (e.g., B1, B2, C1, D1, and D2) can be determined.

FIG. 2 is a flow chart for a simple algorithm to randomly select splice points for a basis set of polynucleotides, and to design oligonucleotides for preparation of chimeric polynucleotides.

FIG. 3 is a flow chart for a simple algorithm to randomly select splice points for a basis set of polynucleotides, and to design double-ended primers for preparation of chimeric polynucleotides. M is the position of the current splice point; h, j and l are the sequence designators; j the sequence position in the alignment; and k is the sequence position in the primer components.

### DETAILED DESCRIPTION OF THE INVENTION

The present invention pertains to methods for generating chimeric polynucleotides, such as polynucleotides encoding polypeptides (“chimeric polypeptides”), using directed evolution of a basis set of polynucleotides.

Basis Set of Polynucleotides

As described herein, a “polynucleotide” is a polymeric chain of nucleotides (e.g., a gene, gene fragment, cDNA, mRNA), and a “polypeptide” is a polymeric chain of amino acids (e.g., a protein). A “basis set” is a group of 2 or more polynucleotides, preferably greater than 3 polynucleotides, such as between 3 and 12 polynucleotides, inclusive; the basis set of polynucleotides is used as the starting materials for the directed evolution. The polynucleotides of the basis set can be of any length; generally, they are greater than 20 nucleotides in length (e.g., approximately 50 nucleotides in length or greater, preferably approximately 75 nucleotides in length or greater, more preferably approximately 100 nucleic acids in length or greater); if desired, only a short fragment of any one of the polynucleotides is used during generation of chimeric polynucleotides. In one embodiment, the basis set comprises at least two polynucleotides that have a high degree of sequence homology or identity; in a preferred embodiment, at least two of the polynucleotides of the basis set have sufficient homology to one another to anneal for priming during polymerase chain reaction. In another embodiment, the basis set comprises at least two polynucleotides that encode polypeptides having structural homology in one or more regions.

To determine the percent homology or identity of two nucleic acid sequences, the sequences are aligned for optimal comparison purposes (e.g., gaps can be introduced in the sequence of one nucleic acid molecule for optimal alignment with the other nucleic acid molecule). The nucleotides at corresponding nucleotide positions are then compared. When a position in one sequence is occupied by the same nucleotide as the corresponding position in the other sequence, then the molecules are homologous at that position. As used herein, nucleic acid “homology” is equivalent to nucleic acid “identity”. The percent homology between the two sequences is a function of the number of identical positions shared by the sequences (i.e., percent homology equals the number of identical positions/total number of positions times 100). In preferred embodiments, at least two polynucleotides in the basis set have at least 50% homology or greater; more preferably, 70% homology or greater; even more preferably, 80% homology or greater; still more preferably, 90% homology or greater. “High” homology, as used herein, refers to 80% homology or greater.

In one embodiment of the invention, one or more of the polynucleotides of the basis set comprise full length genes. A “gene,” as used herein, refers to a specific sequence of nucleotides (e.g., DNA or RNA), typically locatable on a chromosome, that encodes a particular polypeptide (e.g., a protein). In another embodiment of the invention, one or more of the polynucleotides of the basis set comprise partial genes (for example, a polynucleotide comprising one or more exons of a gene). In still another embodiment of the invention, the polynucleotides of the basis set comprise synthetic nucleotide sequences.

The polynucleotides of the basis set can include naturally-occurring nucleic acids (e.g., nucleic acids that are found in an organism, for example, genomic DNA, complementary DNA (cDNA), chromosomal DNA, plasmid DNA, mRNA, tRNA, and/or rRNA). The polynucleotides can also comprise modified nucleic acids. “Modified” nucleic acids include, for example, nucleic acids which are naturally-occurring, as described above, but are modified to alter (e.g., add, delete, or modify) one or more nucleotides. In another embodiment, the polynucleotides of the basis set can include synthetic nucleic acids, including but not limited to, nucleic acids prepared on solid phases using well-known and/or commercially-available procedures, e.g., using an automated nucleic acid synthesizer. In yet another embodiment, a combination of more than one type of nucleic acid can be present (e.g., naturally-occurring and/or modified and/or synthetic nucleic acids). If desired, the naturally-occurring, modified and/or synthetic nucleic acids can comprise modified nucleotides. As used herein, a modified nucleotide is a nucleotide that has been structurally altered so that it differs from a naturally-occurring nucleotide.

The polynucleotides of the basis set can be obtained from various biological and/or chemical materials using standard procedures. For example, naturally-occurring polynucleotides (e.g., genes) can be obtained from organisms, tissues, and/or cells from veterinary or human clinical test samples collected for diagnostic and/or prognostic purposes. For example, cells can be lysed and the resulting lysate can be processed using techniques familiar to one of skill in the art to obtain an aqueous solution of nucleic acid (e.g., DNA and/or RNA) (see, for example, Ausebel, F., et al., Current Protocols in Molecular Biology, Wiley, N.Y. (1988); Maniatis, et al., Molecular Cloning: A Laboratory Manual, Cold Spring Harbor Laboratory, Cold Spring Harbor, N.Y. (1982)). Nucleic acids, where appropriate can also be cleaved to obtain a fragment that contains a desired polynucleotide, for example, by treatment with a restriction endonuclease or other site-specific chemical cleavage methods. Polynucleotides can also be synthesized from nucleotide monomers, e.g., using an automated nucleic acid synthesizer, or can be obtained using recombinant DNA methodology.

If desired, the polynucleotides of the basis set can be modified by introducing features that will facilitate directed evolution. For example, common restriction sites recognized by particular enzymes can be introduced into a polynucleotide by standard techniques (e.g., site directed mutagenesis, such as by PCR-based mutation). An “introduced” or “non-native” restriction site, as used herein, is a restriction site that is incorporated into a polynucleotide at a point where a restriction site was not previously present, or at a point where the alignment had natural homology insufficient for cross-sequence priming. For example, a different restriction site (e.g., a restriction site recognized by a different enzyme) was previously present can be incorporated. In a preferred embodiment, the restriction sites can be introduced without affecting the amino acid sequence encoded by the polynucleotide, due to the degeneracy of the code. A common restriction site can be, for example, a short region suitable for priming, such as a designated splice position from one sequence which is used to replace its cognates in all the other polynucleotides in the basis set.

Design of Chimeric Polynucleotides: “In Silico” Preparation

In the methods of the invention, chimeric polynucleotides are designed, based on the polynucleotides of the basis set. A “chimeric polynucleotide,” as used herein, is a polynucleotide that contains fragments from at least two of the polynucleotides in the basis set. In a preferred embodiment, the chimeric polynucleotide contains one or more fragments from each polynucleotide in the basis set. A “fragment” of a polynucleotide, as used herein, is less than the whole polynucleotide: for example, if a polynucleotide in the basis set is 300 nucleotides in length, a fragment of that polynucleotide comprises from 1 to 299 consecutive nucleotides of the polynucleotide. Usually, the fragment will contain that part of the polynucleotide that is between two splice points in the polynucleotide, or that part of the polynucleotide that is between an end (i.e., a 5′ or 3′ end) of the polynucleotide and a splice point in the polynucleotide. A “splice point” in a polynucleotide is the location at which the polynucleotide is fragmented.

To generate chimeric polynucleotides, splice points of interest within the polynucleotides of the basis set are identified. Each polynucleotide in the set will have the same number of splice points in silico, although not all of the fragments between splice points need be used when generating chimeric polynucleotides in vitro. In one embodiment, an algorithm which defines and aligns natural splice points within the polynucleotides of the basis set is used. In another embodiment, an algorithm which selects random splice points, is used. As used herein, the term “algorithm” refers to step-by-step procedure for solving a problem (e.g., the identification of splice points) in a finite number of steps that frequently involves repetition of an operation, preferably (though not necessarily) with the assistance of computer.

In either embodiment, the algorithm can incorporate desired parameters, including: the number of splice points desired and alignment of the sequences in the basis set. In additional embodiments, the algorithm can further include parameters relating to a desired distance between splice points (e.g., approximately 8-20 base pairs apart, to facilitate PCR priming); if desired, the algorithm can additionally include parameters relating to melting temperatures of hybridized fragments of the polynucleotides of the basis set (e.g., Tmax and Tmin; for example, a Tm between about 50-75° C., inclusive).

If desired, a preliminary step can be added in which splice points are identified which lie in regions of interest in the polynucleotide sequences of the basis set (e.g., regions in which the homology is favorable for hybridization during polymerase chain reaction (PCR)). A pairwise sliding box investigation of the number of exact matches can be formed; this will be quicker than the calculation using Tm, because no floating point calculations are needed. Sequence regions of low utility could be discarded from the areas used for splice points, and sequences of low utility within a specified fragment could also be discarded. Splice points within the homologous regions could then be identified without searching the entire alignment. This preliminary step is particularly useful for constructing chimera using PCR (as described below), for example, when the basis set comprises a set of overlapped oligonucleotides taken from a superfamily alignment; some sequences might contribute only one oligonucleotide, corresponding to a short fragment of a polynucleotide, to the set of chimera.

Alternatively or in addition, if desired, the algorithm can incorporate “weighing” or “biasing” factors. In one embodiment, favorable regions for splice points can be identified using a specified region or a specified number or exact matches in a specified region as a cutoff criterion. For example, the biasing factors can be set so that specific splice points (such as those near the beginning or the end of the polynucleotide) can be rejected. Sets of splice points within specified regions can be identified from Tm calculations, and other sequences added to the natural sets by incremental adjustment of each polynucleotide in the basis set until Tmin is reached with the consensus sequence of the natural set. In one embodiment, the Tm is set to be approximately 50-75° C., inclusive; this will typically correspond to hybridizing of 14-20 base pair regions with about 2 mismatches. The weighing factors can be designed to bias the selection of splice points in regions of the polynucleotides of the basis set that have particular homology (e.g., high homology, or low homology); alternatively or in addition, the weighing factors can incorporate structural “mask” for selection of splice points, which will bias the selection of splice points in structurally identifiable regions of the polypeptides encoded by the polynucleotides of the basis set (e.g., intervening regions; loops; transmembrane sequences; domain or subdomain boundaries; borders and internal divisions of binding sites for cofactors, ligands, prosthetic groups; and borders and internal divisions for control elements, etc.).

For example, in one embodiment of an algorithm, starting with the polynucleotides of the basis set, a sequence alignment in array form A(i,j), where i is the number of the polynucleotide and j the sequence position in the alignment; A(i,j) can be a base character or a blank. A sliding box algorithm brings a box of width n down the alignment, calculating the melting temperature T (i,j) for all base pairs at each position. This calculation can include mismatches, if desired. If a majority of the T(i,j) is high, n is decreased and the T(i,j) are recalculated until the maximum number are between specified limits of Thot and Tcold. The number of T(i,j)s within the limit is stored, along with the initiation point and the box size. The best m overlaps can be reported. This method works particularly well for basis sets having highly homologous sequences.

In another embodiment of an algorithm, the algorithm calculates all the pairwise values for Tmax and Tmin for T(n,m,k,l) between sequences n and m for fragments beginning at position k and extending l bases. Every T(n,m,k,l) between Thot and Tcold generates a pair a(i,j,n) and a(i′,j′,m) corresponding to the fragments in sequences n and m for which it was calculated.

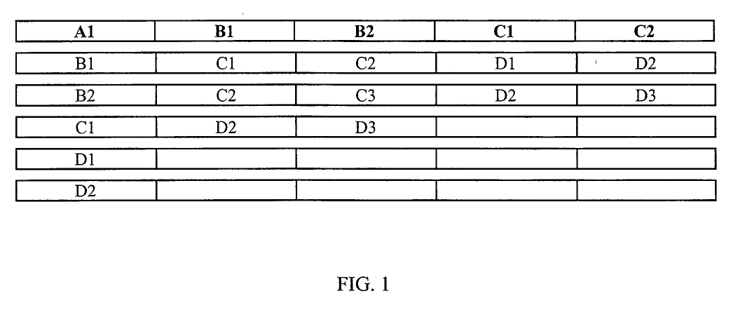

Every a(i,j,n) can be represented as an element A1, and a table can be constructed using the pairs. For example, as shown in FIG. 1, the element A1 hybridizes with the desired melting temperature to B1, B2, C1, D1, and D2 (all the A elements would have the same n value, and for each table all the A elements would start at the same position but be of different lengths). In addition, B1 hybridizes as desired with C1, C2 and D2, and so on. A fully connected set of elements Aw, Bx, Cy and Dz is generated such that Bx, Cy and Dz all appear under Ay, Cy and Dz appear under Bx, and Dz appears under Cy.

This method can be performed using by a tree algorithm in which each branch originating in column A1 is followed to completion. For example, B1 can be followed to C1, which is also found in A1. C1 is followed to D1, which is found in A1 but not in B1. The missing element in B1 generates a penalty of 1 for this branch. The next branch to be investigated extends from A1 to B1 to C1 to D2, which is found in A1 and B1 as well. There is no penalty, since the fragments represented by the elements can span the sequence set at this position. There will not always be such a set of elements at an arbitrary position, so the set of elements, and hence fragments, with the lowest penalty at each position is recorded along with its penalty score. An arbitrary number of “best” splice points can be reported. If no zero or single penalty sets are identified for a particular sequence position, a second table can be constructed starting with the B elements to identify potential sets missing only the A element, etc., until a specified cutoff is reached. In one embodiment, a preliminary step as described above, in which splice points are identified which lie in regions of interest in the polynucleotide sequences of the basis set (e.g., regions in which the homology is favorable for hybridization during polymerase chain reaction (PCR), can be added to this algorithm.

In a third embodiment of the algorithm, a heuristic algorithm can be used for the identification of overlapping oligonucleotide sets in the basis set of polynucleotides, in order to prepare chimeric oligonucleotides as described in detail below. This algorithm begins by identifying favorable regions in an alignment using the number of exact matches in a specified region as the cutoff criterion, as in the preliminary step described above. ‘Natural’ sets within these regions are identified from Tm calculations, and other sequences are added to the natural sets by incremental adjustment of each sequence to be added until T_{low} is reached with the consensus sequence of the natural set. For example, one sequence can be assigned as the master sequence at each spice point; this can be done by arbitrary assignment, or by choosing the sequence with the best local overlap with other members of the set. Sequences with low annealing temperatures can be forced to anneal by progressively substituting codons from the master sequence for mismatched codons. This minimal approach preserves maximum diversity at spice points; in extreme cases complete substitution at a splice point can be used to force annealing between previously unrelated oligonucleotides. This algorithm is particularly useful for basis sets of polynucleotides having low homology with one another, as it assists in the construction of a set of overlapped oligos in which the original gene sequences have been modified to produce favorable overlaps for polymerase chain reaction (PCR). The chimeric polynucleotides prepared by these methods have a much higher diversity than would be produced by random breakage or restriction, since overlaps among the polynucleotides of the basis set are optimized.

Once splice points are identified, chimeric polynucleotides can be generated using a variety of methods presented below. Representative algorithms for identifying splice points are described. In certain embodiments, combinatorial synthesis, or polymerase chain reaction-based synthesis using double primers, can be used.

Preparation of Chimeric Polynucleotides: Splice Point Selection

In one embodiment of the invention, a sequence alignment A(i,j) as described above uses a set of homologous polynucleotides as the basis set for chimera formation. In the most basic variant, the number of splice points desired is specified, and the splice points are chosen by repeated random selection without replacement. The basic selection mechanism is the use of a random number generator to yield a position in amino acid space, followed by multiplication by three to convert to nucleotide space at codon boundaries (as described in detail below). An alignment A(i,j) where i is the sequence designator and j the position is used. For a set of ordered splice points M(h) the chimeric sequences are generated by combinatorial concatenation so that to each vector component Pre(i,j) (j=1,M(1)−1) I vectors are formed by adding the strings A(i,j) (j=M(1),M(2)−1). All the available components are concatenated with all the existing vectors at each splice. For example, starting with the I strings Pre(i,j), a set of 10 sequences would have 10 pre components, 100 chimera after the first splice, etc., forming 10⁶ vectors after five splices. Splice points can generally be constrained to be a sufficient length apart (e.g., at least 12-20 bp) apart to allow for PCR priming; this can be done by discarding random selections which do meet the specified criteria. Alternatively, splice points closer than this can be allowed but treated differently than well spaced splices.

Preparation of Chimeric Polynucleotides by Combinatorial Synthesis

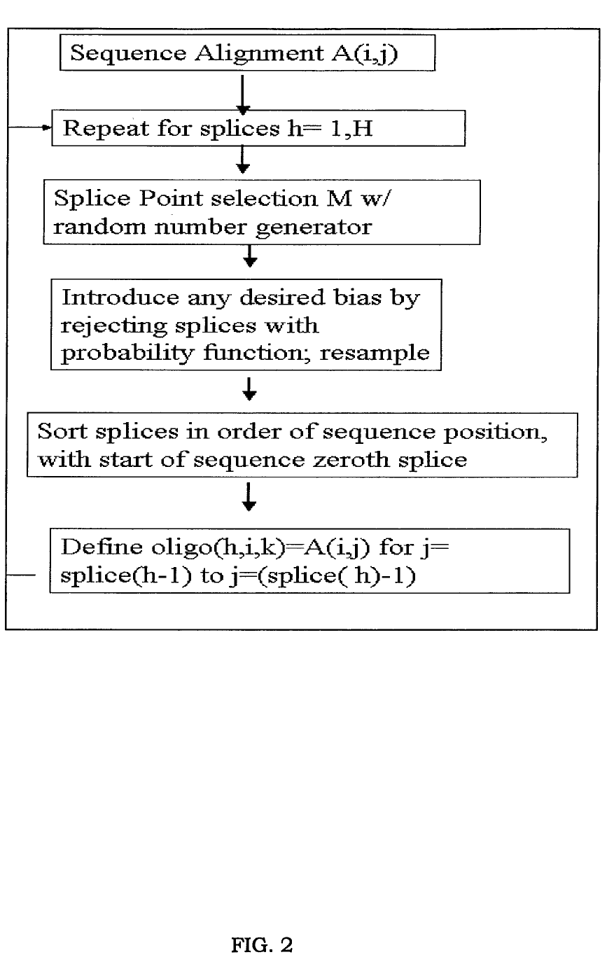

In another embodiment of the invention, generation of polynucleotide chimera is conducted by combinatorial synthesis. In a representative algorithm for combinatorial synthetic methods, the identification of oligonucleotides begins with the alignment A(i,j), and a random number generator is a convenient method of splice point selection. For combinatorial synthesis the oligonucleotides have no overlap, in contrast with double ended primers which connect sequence regions in different polynucleotides (as described below). The algorithm for combinatorial synthesis need only specify the nucleotide sequence in each fragment between splice points. Starting at one end, a set of i polynucleotides gives i fragments for the region between the start and the first oligonucleotide, i more for the region between the first splice point and the second, and so on. If an immobilized synthesis strategy is used (as described below), a linker will be specified for either the 3′ or 5′ set of fragments. A representative algorithm is depicted in FIG. 2.

Using the methods described above, a set of splice points is defined in polynucleotides of the basis set, such that a desired number of fragments (“M”) between the splice points will be produced to use as the building blocks for the chimeric polynucleotides. The M fragments are numbered consecutively for each polynucleotide in the basis set (e.g., consecutively from 5′ to 3′). Thus, each polynucleotide in the basis set will have “corresponding fragments,” which are the fragments in each polynucleotide that have the same number. In a preferred embodiment, the combinatorial synthesis is used for basis sets comprising synthetic polynucleotides; in another preferred embodiment, the combinatorial synthesis is used for basis sets comprising polynucleotides that contain gene fragments (i.e., less than an entire gene).

For a basis set containing N polynucleotides, having M number of fragments, a set of non-overlapping oligonucleotides are prepared for each of the M fragments. An “oligonucleotide,” as used herein, refers to a chain of nucleotides, generally short in length (e.g., less than 40 nucleotides, preferably less than 30 nucleotides, even more preferably less than 20 nucleotides). Each individual oligonucleotide comprises nucleic acids hybridizing to a selected fragment. The oligonucleotides form a non-overlapping set: that is, none of the oligonucleotide hybridize to the same regions within any one polynucleotide of interest. These oligonucleotides are not primers, since they have no overlap and do not anneal, but are instead combinatorially combined (e.g., by ordered ligase reactions, as described herein).

To perform combinatorial synthesis of chimeric polynucleotide, stepwise amplification and ligation (joining) of the M fragments, correctly ordered, for each of the N polynucleotides in the basis set is performed. In one embodiment, the oligonucleotides are combined (ligated) stepwise (one at a time) by location. In another embodiment, the oligonucleotides are combined pairwise by location. Fragments are “correctly ordered” when they are sequentially attached in the order corresponding to the number M of the position of the fragments in each polynucleotide: (e.g. the first fragment followed by the second fragment, the fifth fragment followed by the sixth fragment). Amplification by PCR can be used to select the correctly ordered pairs (e.g., M1M2, rather than M2M1); alternatively, the correctly ordered pairs can also be selected by a blocking/unblocking strategy, without use of PCR. The oligonucleotides corresponding to two consecutive fragments of the M fragments of each of the N polynucleotides (e.g., M1 and M2) are mixed and randomly ligated. Selective amplification of the correctly ordered sets of fragments (e.g., dimers of M1 and M2) can be can be performed, using forward primers that hybridize to the 5′ ends of the first fragments, and reverse primers that hybridize to the 3′ ends of the second of the M fragments. This process is repeated for other sets of fragments (e.g., the third and fourth fragments of the N polynucleotides, the fifth and sixth fragments of the N polynucleotides, etc.). The correctly ordered sets of oligonucleotides produced by ligation of fragments (e.g., 1, 2 dimers formed by ligation of the first and second fragments) are mixed with the correctly ordered sets produced by the ligation of the subsequent sets of oligonucleotides (e.g., 3, 4 dimers formed by ligation of the third and fourth fragments), and randomly ligated. The correctly ordered sets (e.g., tetramers of M1, M2, M3 and M4) can then be selectively amplified by PCR using the forward primers for the 5′ end of the first fragment (e.g., M1) and the reverse primers for the 3′ end of the last fragment (e.g., M4). Alternatively, as indicated above, blocking and unblocking strategy can be used in lieu of PCR.

The larger order combinations (e.g., tetramer (M1, M2, M3, M4), tetramer (M5, M6, M7, M8)) are mixed and ligated. Correctly ordered chimeric polynucleotides are selectively amplified by PCR using forward primers for the 5′ end of the first fragment and reverse primers for the 3′ end of the last fragment. As a result, a multitude of correctly ordered chimeric polynucleotides, comprising a fragment from each of the N polynucleotides, is generated.

Preparation of Chimeric Polynucleotides I: Polymerase Chain Reaction (PCR)-Based Methods Using Double Primers

In another embodiment of the invention, generation of polynucleotide chimera is conducted by preparation of oligonucleotide “double primers” based on splice points. “Primers” are oligonucleotides that hybridize in a base-specific manner to a complementary strand of nucleic acid molecules. Such probes and primers include polypeptide nucleic acids, as described in Nielsen et al., Science, 254, 1497-1500 (1991). In a preferred embodiment, a “primer” refers in particular to a single-stranded oligonucleotide which acts as a point of initiation of template-directed DNA synthesis using well-known methods (e.g., PCR, LCR) including, but not limited to those described herein. In a representative algorithm for double primer methods, the starting point of the algorithm is the alignment A(i,j) as previously described. It is not necessary to give the sequences of all the chimeric products to describe the primers, nor is it always desirable to do so because of the very large number of chimera which can be. In addition to A(i,j) i=1,I and j=1,J the number of splice points H and any biasing information which is desired, is included in the algorithm.

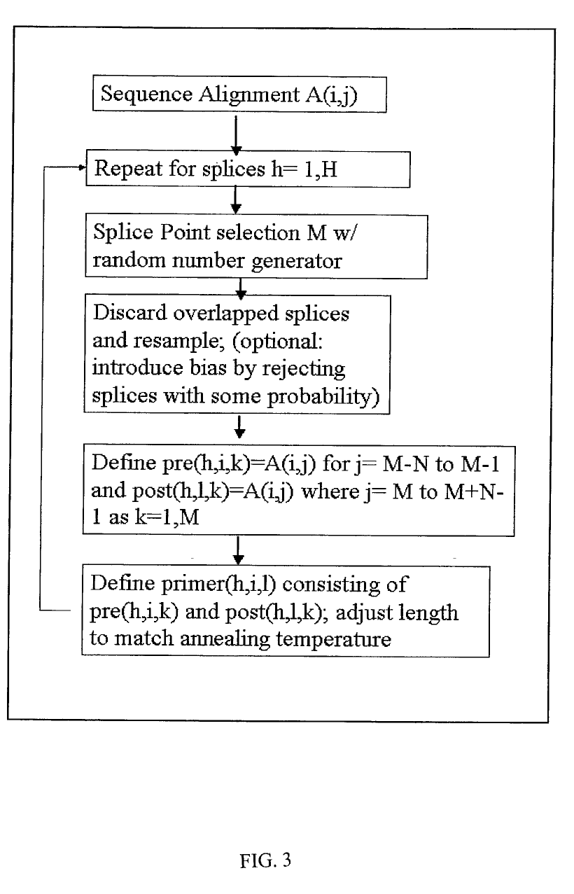

As indicated in FIG. 3, each double primer in a double primer set comprises two regions (a “pre” and a “post” region): an oligonucleotide primer region for a polynucleotide in the basis set (“pre” region), joined to and followed immediately by an oligonucleotide primer region for the complement of that splice point for another polynucleotide in the basis set (“post” region). The double primers at each splice point M(h) are formed by the combinatorial concatenation of the pre and post subsequences. Better matches can be obtained by calculating the Tm for each pre and post with its complement and adjusting them by stepwise lengthening or shortening until the closest value to a desired Tm can be obtained for annealing of the entire primer to each gene. Gap characters in A(ij) can be skipped so that pre and post are M characters in length before Tm adjustment.

Variations on this method can include biasing the selection to make the splice points more evenly spaced, or to make it probable that they be located in regions of high or low homology. Splice points can be concentrated in selected regions (e.g., loop regions or, conversely, regions of conserved secondary structure) or forbidden to lie in other regions, or a region in one of the sequences could be specified as an obligatory component of all of the chimera. In an extreme case, most of the chimera sequences can be constrained to be derived from a single polynucleotide in the basis set, and short elements can be swapped in at selected positions from other (e.g., homologous) polynucleotides in the basis set. Biasing can be performed at the level of checking for overlapped splices.

Overlapped splice regions can be discarded or given an alternative treatment because of hybridization possibilities between subsequences designed to prime basis set sequences and chimeric regions not present in the basis set. The most economical approach, other than the discard option, treats new splices with overlapped primer regions as alternative versions of the previous overlapped splice; a chimeric sequence could include a primer from the splice 2 set or the splice 2a set, but not both.

Using the methods described above, a set of splice points is defined in the polynucleotides of the basis set. For each splice point, an oligonucleotide double primer set is generated, so that the set of double primers includes double primers comprising all possible combinations of pre and post regions for each splice point. Using simple forward and reverse primers for each polynucleotide in the basis set, and a set of double primers for each splice point, a full set of chimera can be generated using polymerase chain reaction techniques. Polymerase chain reaction techniques are well known in the art (see, e.g., U.S. Pat. Nos. 4,683,202, 4,683,195, 4,965,188, and 4,683,202). The entire teachings of these patents are incorporated by reference herein.

Modifications to the Methods of Preparing Chimeric Polynucleotides

If desired, a solid phase can be used for attachment of the components during synthesis of the chimeric polynucleotides. The solid phase can be a solid medium, such as a microtiter plate, a membrane (e.g., nitrocellulose), a bead, a dipstick, a thin-layer chromatographic plate, a pin, a chip, or other solid medium. Attaching a 5′ portion of the first fragment (M1) to a solid phase allows the combinatorial construction of a correctly ordered library of chimeric polynucleotides, because sequential ligation of fragments can be performed. In one embodiment, for combinatorial methods as described above, a strategy can be used in which only one 5′-3′ bond can be formed between any two fragments because of phosphorylation state, chemical modification, or attachment to a solid support at (at least) one end of one of the fragments. For example, if M1 fragments are attached at one end to a solid support, combinatorial ligation of the M1 and M2 fragments can yield only correctly ordered M1-M2 pairs. Addition of the M3 fragments to the attached M1-M2 pairs followed by ligation will then yield only M1-M2-M3 triplets, etc.

Optional “Cleaning” Steps to Concentrate Chimeric Polynucleotides of Interest

If desired, a “polishing” step can be incorporated during synthesis of the chimeric polynucleotides by the methods described above. In a “polishing” step, loose single stranded ends of PCR products are briefly digested with an exonuclease digestion (e.g., at low enzyme activity). Such digestion removes many of the obstacles to polymerase and nick repair, and can be advantageous when mismatches occur at the end of a primer segments.

Alternatively or in addition, if desired, unwanted PCR intermediates can be eliminated during synthesis of the chimeric polynucleotides, through the use of “poisoned primers”. A “poisoned primer” is a primer (nucleic acid) which hybridizes with high stringency to an intermediate which is incapable of supporting PCR, thereby interrupting extension between a viable forward primer and a viable reverse primer. For example, a modification of the 3′ end of a primer which prevents hybridization (e.g., addition of a non-homologous tail such as polyA) can be used. A small number of poisoned primers can often remove a large number of sequences from the pool of polynucleotides available for PCR.

The chimeric polynucleotides can be separated and characterized using standard techniques. For example, in one embodiment, MALDI-TOF mass spectroscopy can be used. MALDI-TOF MS allows biological polymers to be studies intact, and can provide accurate mass resolution to characterize the chimera distribution produced herein (see, e.g., Ross, P. L. et al., Anal. Chem. 70(10):2067-73 (1998)).

Production and Selection of Desired Polynucleotides

The chimeric polynucleotides can then be expressed, using standard techniques. For example, the chimeric polynucleotides can be introduced into a host cell for expression (see, e.g., Huse, W. D. et al., Science 246: 1275 (1989); Viera, J. et al., Meth. Enzymol. 153: 3 (1987)). The chimeric polynucleotides can be expressed, for example, in an E. coli expression system (see, e.g., Pluckthun, A. and Skerra, A., Meth. Enzymol. 178:476-515 (1989); Skerra, A. et al., Biotechnology 9:23-278 (1991)). They can be expressed for secretion in the medium and/or in the cytoplasm of bacteria (see, e.g., Better, M. and Horwitz, A., Meth. Enzymol. 178:476 594(1989)); alternatively, they can be expressed in other organisms such as yeast or mammalian cells (e.g., myeloma or hybridoma cells). One of ordinary skill in the art will understand that numerous expression methods can be employed to produce chimeric polypeptides, encoded by the chimeric polynucleotides described herein. By fusing the chimeric polynucleotides to additional genetic elements, such as promoters, terminators, and other suitable sequences that facilitate transcription and translation, expression in vitro (ribosome display) can be achieved. Similarly, Phage display, bacterial expression, baculovirus-infected insect cells, fungi (yeast), plant and mammalian cell expression can be obtained.

Selection of chimeric polypeptides of interest can subsequently be performed by conducting assays to identify those chimeric polypeptides having a desired activity or function. The chimeric polypeptides can be screened by appropriate means for particular polypeptides having specific characteristics. For example, catalytic activity can be ascertained by suitable assays for substrate conversion and binding activity can be evaluated by standard immunoassay and/or affinity chromatography. Assays for these activities can be designed in which a cell requires the desired activity for growth. For example, in screening for polypeptides that have a particular activity, such as the ability to degrade toxic compounds, the incorporation of lethal levels of the toxic compound into nutrient plates would permit the growth only of cells expressing an activity which degrades the toxic compound (Wasserfallen, A., Rekik, M., and Harayama, S., Biotechnology 9: 296-298 (1991)). Chimeric polypeptides can also be screened for other activities, such as for an ability to target or destroy pathogens. Assays for these activities can be designed in which the pathogen of interest is exposed to the chimeric polypeptides, and those polypeptides demonstrating the desired property (e.g., killing of the pathogen) can be selected.

The following Exemplification is offered for the purpose of illustrating the present invention and are not to be construed to limit the scope of this invention. The teachings of all references cited are hereby incorporated herein in their entirety.

### Exemplification

A. Material and Methods

The methods described herein are used to evaluate chimeric polypeptides from two systems: the small heat shock protein superfamily and the control system in nitric oxide synthase.

Previous experiments within the small heat shock protein superfamily, in which the N terminal region was swapped, demonstrated N terminal aggregation control and produced molecular chaperones with novel properties. There are four major regions within sHSP superfamily proteins; the N and C termini, involved in high level aggregation (N) and tetramer formation/chaperonin-like activity (C), the common core domain, and the extended β6 loop, involved in dimer formation. These regions can be considered in selection of splice points that are used in combinatorial synthesis of chimera from a set of basis genes.

Starting materials include a basis set consisting of four small heat shock protein superfamily genes; two (aA and aB crystalline) are highly homologous (>80% with many regions of identity or near identity), while two others (plant and bacterial sequences) are of low homology for PCR purposes and could not be shuffled by existing methods of directed evolution. Primers include four forward and four reverse primers corresponding to the ends of the four genes with extensions for insertion into cloning and expression vectors, and twelve double ended primers at each splice point for chimera generation. Each primer is designed to anneal to at least two genes at regions adjacent to a splice point with a Tm or 65-70° C. An additional four primers at each splice point span the splice point on a gene.

Trials are conducted with two genes and one splice point and in more complex systems up to four genes and four splice points to examine the diversity and completeness of the chimera set formed. PCR is performed using pfu turbo polymerase in a Techne Genius thermocycler. Two strategies are compared: thirty cycles with all genes and primers, and sequential PCR. Sequential PCR starts with a few linear cycles with the forward primers and genes only. After addition of the first splice point primers, a few cycles (3-5) of PCR are run and the next set of primers added. The procedure is repeated until all desired splices are included, and the reverse primers are added to complete the synthesis with a few cycles of PCR. Simulations indicate that this method produces a more even distribution of products.

Sets of chimera are evaluated by electrophoresis, restriction analysis, and MALDI-TOF Mass spectroscopy. In the simplest cases, two chimera are generated from two basis set genes; these are readily detectable with electrophoresis, since some of the genes have different length 3′ and 5′ terminal extensions. Intermediate cases can be evaluated by using natural restriction sites to differentiate between chimera of similar length. The population generated by four genes and four splice points includes n^{(m+1)} or 1024 chimera. Individual components can be characterized in the distribution by mass spectroscopy. The results can be simplified by using different restriction enzymes to eliminate subsets of chimera from the samples if desired.

For example, experiments that use a set of four sHSP genes with three splice sites produce 256 chimera; this set is large enough to be systematic, but small enough so that all ‘successful’ (well expressed) chimera can in principle be subjected to preliminary evaluation for aggregate size and activity. The set of chimeric genes will be small enough for evaluation by MALDI-TOF.

In addition to the rational selection of splice points to produce the limited chimera set described above, the sHSP superfamily is used in extensive experiments using the methods described herein. The sHSP superfamily is a good choice for this because the genes are small, the potential basis set is extensive, and potential selection criteria are available (temperature resistance, stabilization of reporter proteins). E. coli expression systems are used for this work initially, although a phage display system in which chimeric genes are expressed as a fusion protein with a viral coat component can also be used (see, e.g., Swimmer, C., et al, PNAS USA 89(9):3750-60 (1992)); this has the advantage of linking the expressed protein to its DNA.

The approaches described herein are used to investigate the control elements in nitric oxide synthases, a family of enzymes which produce nitric oxide as a molecular signal in the central nervous system, in the control of vascular tone (blood pressure), and in many other physiologically important signal transduction pathways. A set of regions involved in control within the sequence of NOS can be shuffled to produce an extended design chimera set analogous to that described above for sHSPs. In addition, random chimera are generated from limited regions in the NOS gene; this approach generates more chimera of interest than chimera generation from the entire NOS gene, which is very large. Chimeric regions are ligated back into full length NOS enzymes to produce the desired set of novel proteins. Designed NOS chimera have already been produced which have altered control properties; and this area could produce signal generators with long range gene therapy potential.

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. A method for generating chimeric polynucleotides, comprising:
a) providing a basis set of polynucleotides, wherein the basis set comprises two or more different polynucleotides;
b) identifying splice points within the polynucleotides of the basis set, wherein each polynucleotide in the basis set has the same number of splice points;
c) generating oligonucleotide double primer sets for each splice point, wherein each double primer in a set comprises a “pre” region joined to and followed immediately by a “post” region, and wherein the “pre” region comprises an oligonucleotide primer for a splice point in one polynucleotide in the basis set, and the “post” region comprises the complement of an oligonucleotide primer for that splice point in another polynucleotide in the basis set, and wherein the set of double primers includes double primers comprising all possible combinations of pre and post regions for each splice point;
d) using the double primer sets in polymerase chain reaction to amplify combinations of fragments;
thereby generating a multitude of chimeric polynucleotides, wherein each chimeric polynucleotide comprises a fragment from at least two of the polynucleotides in the basis set.

2. The method of claim 1, wherein the basis set comprises more than two different polynucleotides.

3. The method of claim 1, wherein at least two of the polynucleotides of the basis set have high homology to one another.

4. The method of claim 1, wherein at least one of the polynucleotides of the basis set comprises a whole gene.

5. The method of claim 4, wherein all of the polynucleotides of the basis set comprise whole genes.

6. The method of claim 1, wherein none of the polynucleotides of the basis set comprises a whole gene.

7. The method of claim 1, wherein at least one of the polynucleotides of the basis set comprises a synthetic nucleic acid.

8. The method of claim 1, wherein the chimeric polynucleotides comprise polynucleotides comprising a fragment from each polynucleotide in the basis set.

9. The method of claim 1, wherein the splice points are identified by use of an algorithm that defines the positions of splice points.

10. The method of claim 9, wherein the splice points are identified by random selection.

11. The method of claim 9, wherein the algorithm incorporates information regarding alignment of the polynucleotides.

12. The method of claim 9, wherein the algorithm defines a desired distance between splice points.

13. The method of claim 9, wherein the algorithm incorporates weighing factors to bias selection of splice points.

14. The method of claim 13, wherein the weighing factors bias selection of splice points in regions of interest in the polynucleotides of the basis set.

15. The method of claim 13, wherein the weighing factors bias selection of splice points in regions having a preselected percentage of homology among the polynucleotides of the basis set.

16. The method of claim 13, wherein the weighing factors bias selection of splice points in structurally identifiable regions of the polypeptides encoded by the polynucleotides of the basis set.

17. The method of claim 1, wherein the chimeric polynucleotides are generated on a solid phase.

18. The method of claim 1, further comprising one or more “polishing” steps during polymerase chain reaction, in which loose single stranded ends of products are briefly digested with an exonuclease.

19. The method of claim 1, further comprising utilizing one or more “poisoned primers” which hybridizes with high stringency to an product which is incapable of supporting polymerase chain reaction, thereby interrupting extension during polymerase chain reaction.

20. A method for generating chimeric polynucleotides, comprising:
a) providing a basis set of polynucleotides, wherein the basis set comprises two or more different polynucleotides;
b) identifying splice points of interest within the polynucleotides of the basis set, wherein each polynucleotide in the basis set has the same number of splice points, and wherein the splice points divide each polynucleotide into M consecutive fragments in a correct order;
c) generating non-overlapping oligonucleotides for each fragment of the M fragments for each polynucleotide in the basis set;
d) ligating oligonucleotides corresponding to consecutive fragments in the correct order;
e) selecting correctly ordered combinations of fragments;
thereby generating a multitude of correctly ordered chimeric polynucleotides, wherein each chimeric polynucleotide comprises a fragment from each of the polynucleotides in the basis set.

21. The method of claim 20, wherein step (d) is performed by:
d1) ligating oligonucleotides corresponding to two consecutive fragments in the correct order;
d2) selecting correctly ordered combinations of fragments;
d3) repeating steps (d1) and (d2) for all sets of two consecutive fragments in the correct order;
d4) mixing and ligating the products of steps (d2) and (d3) in the correct order,
thereby generating a multitude of correctly ordered chimeric polynucleotides, wherein each chimeric polynucleotide comprises a fragment from each of the polynucleotides in the basis set.

22. The method of claim 20, wherein the basis set comprises more than two different polynucleotides.

23. The method of claim 20, wherein at least two of the polynucleotides of the basis set have high homology to one another.

24. The method of claim 20, wherein at least one of the polynucleotides of the basis set comprises a whole gene.

25. The method of claim 24, wherein all of the polynucleotides of the basis set comprise whole genes.

26. The method of claim 20, wherein none of the polynucleotides of the basis set comprises a whole gene.

27. The method of claim 20, wherein at least one of the polynucleotides of the basis set comprises a synthetic nucleic acid.

28. The method of claim 20, further comprising introducing at least one non-native restriction point into at least one polynucleotide of the basis set.

29. The method of claim 20, wherein the chimeric polynucleotides comprise polynucleotides comprising a fragment from each polynucleotide in the basis set.

30. The method of claim 20, wherein the splice points are identified by use of an algorithm that defines the positions of splice points of splice points.

31. The method of claim 30, wherein the splice points are identified by random selection.

32. The method of claim 30, wherein the algorithm incorporates information regarding alignment of the polynucleotides.

33. The method of claim 30, wherein the algorithm defines a desired distance between splice points.

34. The method of claim 30, wherein the algorithm incorporates weighing factors to bias selection of splice points.

35. The method of claim 34, wherein the weighing factors bias selection of splice points in regions of interest in the polynucleotides of the basis set.

36. The method of claim 34, wherein the weighing factors bias selection of splice points in regions having a preselected percentage of homology among the polynucleotides of the basis set.

37. The method of claim 34, wherein the weighing factors bias selection of splice points in structurally identifiable regions of the polypeptides encoded by the polynucleotides of the basis set.

38. The method of claim 20, wherein the chimeric polynucleotides are generated on a solid phase.

39. The method of claim 20, further comprising utilizing one or more “poisoned primers” which hybridizes with high stringency to an product which is incapable of supporting polymerase chain reaction, thereby interrupting extension during polymerase chain reaction.

40. The method of claim 21, wherein in step (d2), correctly ordered combinations of fragments are selected by selective polymerase chain reaction amplification.

41. The method of claim 21, wherein in step (d2), correctly ordered combinations of fragments are selected by blocking of incorrectly ordered combinations of fragments.
